(19) United States
(12) Patent Application Publication
(10) Pub. No.: US 2025/0246273 A1
(43) Pub. Date: Jul. 31, 2025

(54) EVALUATION METHOD, VISUALIZATION METHOD, EVALUATION DEVICE, AND VISUALIZATION DEVICE

(71) Applicant: FUJIFILM Corporation, Tokyo (JP)

(72) Inventor: Ryo SHIMURA, Ashigarakami-gun (JP)

(73) Assignee: FUJIFILM Corporation, Tokyo (JP)

(21) Appl. No.: 19/083,235

(22) Filed: Mar. 18, 2025

Related U.S. Application Data
(63) Continuation of application No. PCT/JP2023/031844, filed on Aug. 31, 2023.

(30) Foreign Application Priority Data
Sep. 21, 2022 (JP) 2022-150222

(51) Int. Cl. G16H 10/40; G16B 45/00; G16B 50/10

(52) U.S. Cl. CPC G16H 10/40; G16B 45/00; G16B 50/10

(57) ABSTRACT

An embodiment according to the technology of the present disclosure provides an evaluation method and an evaluation device for evaluating biological robustness of a cluster of multi-omics data and of clustering, and a visualization method and a visualization device for a result of the evaluation. According to an aspect of the present invention, there is provided an evaluation method for a cluster executed by an evaluation device including a processor. The evaluation method includes causing the processor to: acquire cluster allocation information obtained from clustering performed on multi-omics data consisting of two or more omics by any method independent for each omics; calculate a rate of match between cluster allocations of different omics for each cluster from the cluster allocation information; calculate a first quantitative index indicating biological robustness of the cluster for each cluster based on the rate of match; and output the calculated first quantitative index.

### CROSS-REFERENCE TO RELATED APPLICATIONS

The present application is a Continuation of PCT International Application No. PCT/JP2023/031844 filed on Aug. 31, 2023 claiming priority under 35 U.S.C § 119(a) to Japanese Patent Application No. 2022-150222 filed on Sep. 21, 2022. Each of the above applications is hereby expressly incorporated by reference, in its entirety, into the present application.

### BACKGROUND OF THE INVENTION

### 1. Field of the Invention

The present invention relates to an evaluation method, a visualization method, an evaluation device, and a visualization device, and particularly relates to a technique for evaluating a cluster of omics data or clustering and a technique for visualizing an evaluation result.

### 2. Description of the Related Art

### [Tasks of Clustering Evaluation]

A clustering technique is a method that classifies a sample population based on a randomly defined similarity to form sets (referred to as clusters) and that is widely used as one of search methods using data analysis.

Clustering is positioned as a kind of unsupervised machine learning and is generally performed in unsupervised setting. Here, “supervised” learning indicates a case where input data and correct answer information of “input data of sample i is X and belongs to cluster K” are given in advance. In contrast, in “unsupervised” learning in which clustering analysis is mainly performed, problem setting is that “classification is performed in a situation in which only the input data X of the sample i is given and it is unclear which cluster is correct”. Hereinafter, it is assumed that clustering is performed in the unsupervised setting in this specification.

A task of the unsupervised clustering is to evaluate an execution result. The evaluation makes it possible to verify whether the obtained cluster is a valid result. As a result of the evaluation, the user performs (1) adjustment of setting values (values that the user generally needs to set; hyperparameters in machine learning) and (2) selection of an appropriate one from a plurality of clustering methods or algorithms.

In (1), it is known that, even though the same clustering algorithm is used, the results greatly differ depending on the setting values. In (2), various clustering methods have been developed so far, and the method with good performance often varies depending on the application and the scale and/or properties of the data. Therefore, the evaluation is also important as one criterion for appropriately selecting the method to be used and the setting values thereof.

In general, in the supervised machine learning, the evaluation is performed by calculating an index (referred to as an external criterion) based on the rate of match between a correct answer label and a predicted label. For example, in a case where a correct answer label {sample 1, sample 2, sample 3, sample 4, sample 5}={A, B, A, C, C} is determined for five samples, the rate of match (correct answer rate) of a predicted label {A, B, B, A, C} is ⅗=0.6. However, in the clustering analysis, the unsupervised setting, that is, the correct answer label is not given. Therefore, it is usually difficult to calculate the index based on the rate of match with the correct answer.

Therefore, various evaluation indexes (referred to as internal criteria) of the clustering results without using the correct answer label have been considered so far. An example of the evaluation index is an index based on a degree of aggregation in the same cluster and a degree of deviation between different clusters. In the above-described case, “clustering in which a distance between sample 1 belonging to a predicted cluster A and sample 4 belonging to the same predicted cluster A is small and a distance between sample 1 belonging to the predicted cluster A and sample 2 belonging to a predicted cluster B is large” is considered to be appropriate clustering. Here, the distance between the samples is a value calculated by observed values.

It has been reported that there is an index that has a good correlation with the external criterion in an experiment using artificially generated data among these internal criteria. However, there are problems, such as the fact that the results are based on artificial data under limited conditions and the fact that the optimal internal criterion varies depending on target data, and there are still many tasks in evaluating the clustering result based on the internal criterion.

### [Clustering of Omics Data]

First, a general term for genes present in a living body is a genome, and data obtained by measuring the genome is called genomic data. In addition, this concept can be extended by being associated with a series of phenomena occurring in the body. In addition to the genome, for example, the following have been proposed: an epigenome which is a general term for acquired regulatory factors that do not involve changes in gene sequences; a transcriptome which is a general term for gene transcripts; and a proteome which is a general term for proteins. In recent years, the concept has emerged in which the “general term for biological substances present in the same hierarchy in a life phenomenon” is generally called omics/omics data.

In a biomedical field, clustering analysis on various types of omics data is actively performed. For example, in a case where transcriptome data of cancer patients is clustered, it is possible to classify the same cancer into subtype groups with different properties, and research or the like is being attempted to select high-accuracy treatment and/or medication suitable for each subtype. Furthermore, in recent years, the concept of multi-omics has emerged, and an attempt has been made to integrate and analyze information obtained from a plurality of omics to more precisely model complex life phenomena.

For example, as the technique related to the clustering, techniques described in the following are known: “A robustness metric for biological data clustering algorithms”, Yuping Lu et al., [Searched on Sep. 9, 2022], Internet (https://pubmed.ncbi.nlm.nih.gov/31874625/); “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A: 1023949509487); and “Multiplatform analysis of 12 cancer types reveals molecular classification within and across tissues of origin”, Katherine A Hoadley et al., [Searched on Sep. 9, 2022], Internet (https://pubmed.ncbi.nlm.nih.gov/25109877/).

[Technique Described in “A robustness metric for biological data clustering algorithms”, Yuping Lu et al., [Searched on Sep. 9, 2022], Internet (https://pubmed.ncbi.nlm.nih.gov/31874625/)].

In “A robustness metric for biological data clustering algorithms”, Yuping Lu et al., [Searched on Sep. 9, 2022], Internet (https://pubmed.ncbi.nlm.nih.gov/31874625/), Lu et al. propose an evaluation index “Robustness” for the robustness of setting values of a clustering algorithm. The term “robust” means response insensitivity of the result of the clustering algorithm to changes in the setting values.

In “Robustness”, clustering is performed on one algorithm with different settings (for example, while changing the number of clusters). As the number of common elements in the results is larger, the robustness is higher. Examples will be described below.
(i) Clustering is performed on three samples 10 times with different settings using an algorithm X.
(ii) In a case where attention is paid to sample a and sample b, it is assumed that the number of times the samples a and b belong to the same cluster is seven. In this case, 7/10=0.7 is established for (a, b).
(iii) Similarly, it is assumed that 5/10=0.5 is established for (b, c), and 0.3 is established for (c, a).
(iv) In this case, the “Robustness” of X is (0.7+0.5+0.3)/3=0.5.

“Robustness” focuses on the selection of an omics data clustering analysis algorithm among the internal criteria. In particular, in a case of an unsupervised data set having a property that is sensitive to setting values as in omics data clustering analysis, it is difficult to determine appropriate setting values. In this situation, a method that is insensitive to the setting values is preferable, and the above can be achieved by selecting an algorithm with high “Robustness”.

On the contrary, since “Robustness” is an index for giving one score to one algorithm, it is not possible to use “Robustness” for the task of the clustering “(1) the adjustment of the setting values”.

[Technique Described in “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A: 1023949509487)].

Another task in the clustering analysis method is clustering stability. Some clustering algorithms use a random number to generate an initial state. This algorithm does not guarantee that cluster allocations obtained each time the algorithm is executed is the same. In solutions obtained by different initial states, there is a high possibility that there will no significant difference between evaluation values. Therefore, even in a case where an appropriate evaluation index is used, it is difficult to determine which execution result is best.

Therefore, there is a method called “consensus clustering” that extracts a common part of a plurality of cluster allocations executed with randomly determined initial values and that derives a final cluster allocation. “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A: 1023949509487) is a representative method and is a method for acquiring a reliable cluster.

A procedure of “Consensus Clustering” is as follows:

1. The following is executed up to h=1, 2, . . . , H
(a) D^{(h)} is subsampled from data D
(b) Clustering is executed on D^{(h)}, and D^{(h)} is substituted into M^{(h)}
(c) M=M∪M^{(h)} is established

2. A consensus matrix M is calculated from M
(a) In a case where the number of samples is N and (i, j) is a set of an i-th sample and a j-th sample, the N×N dimensional matrix M^{(h)} storing execution results of h-th clustering is defined by the following Expression (1).

$${M}^{( h )} ( i , j ) = \{ \begin{matrix}1 if ( i , j ) is in same cluter \\ 0 otherwise\end{matrix} \qquad ( 1 )$$

(b) In addition, an N×N dimensional matrix I^{(h)} storing whether the sample (i, j) is included in D^{(h)} subsampled in the procedure 1-(a) is defined by the following Expression (2).

$${I}^{( h )} ( i , j ) = \{ \begin{matrix}1 if both samples ( i , j ) are included in {D}^{( h )} \\ 0 otherwise\end{matrix} \qquad ( 2 )$$

(c) In this case, the consensus matrix M is represented by the following Expression (3).

$$M ( i , j ) = \frac{{\sum }_{h} {M}^{( h )} ( i , j )}{{\sum }_{h} {I}^{( h )} ( i , j )} \qquad ( 3 )$$

Finally, clustering in a case where the value of the consensus matrix calculated by the above procedure is regarded as a distance between samples is executed to obtain a final clustering allocation (where it is converted into 1−M). [Technique Described in “Multiplatform analysis of 12 cancer types reveals molecular classification within and across tissues of origin”, Katherine A Hoadley et al., [Searched on Sep. 9, 2022], Internet (https://pubmed.ncbi.nlm.nih.gov/25109877/)].

In “Multiplatform analysis of 12 cancer types reveals molecular classification within and across tissues of origin”, Katherine A Hoadley et al., [Searched on Sep. 9, 2022], Internet (https://pubmed.ncbi.nlm.nih.gov/25109877/), “Consensus Clustering” described in “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A:1023949509487) is applied to multi-omics data.

First, clustering is performed in each omics (the clustering algorithm follows the previous studies), and the results of the clustering are converted into binary vectors. Then, the vectors are combined to generate a matrix of dimensions of (the sum of the numbers of clusters of each omics)×the number of samples.

Specifically, it is assumed that the samples belonging to a first cluster of omics 1 are samples 1, 2, and 5, which is expressed as “omics 1-c1 =[1, 1, 0, 0, 1]”. Now, in a case where there are three omics, each cluster allocation can be represented as follows:

Omics 1-c1 =[1, 1,0,0, 1]

Omics 1-c2 =[0, 1, 0, 1, 0]

Omics 1-c3 =[1, 0, 0, 1, 0]

Omics 2-c1 =[1, 0, 1, 1, 1]

Omics 2-c2 =[0, 1, 1, 0, 0]

Omics 3-c1 =[1, 0, 0, 1, 1].

In the matrix obtained by combining these cluster allocations, each row can be regarded as a “feature amount of a cluster”. “Consensus Clustering” described in “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A:1023949509487) is performed using this matrix as an input. A “cluster of clusters” obtained by combining similar clusters among the clusters extracted in each omics is finally obtained.

### [Clustering Method in Multi-Omics Data Analysis]

Further, clustering analysis is used in multi-omics data analysis, in addition to “A robustness metric for biological data clustering algorithms”, Yuping Lu et al., [Searched on Sep. 9, 2022], Internet (https://pubmed.ncbi.nlm.nih.gov/31874625/), “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A:1023949509487), and “Multiplatform analysis of 12 cancer types reveals molecular classification within and across tissues of origin”, Katherine A Hoadley et al., [Searched on Sep. 9, 2022], Internet (https://pubmed.ncbi.nlm.nih.gov/25109877/). So far, a plurality of clustering methods specialized for multi-omics data analysis have been developed. These clustering methods are not techniques for the internal criterion, but are equally effective in obtaining more valid (especially from a biological perspective) clusters.

### [Generalization of Concept of Multi-Omics]

The concept of multi-omics is generally referred to as “multi-view” or “multi-modal” in the field of machine learning (omics=view/modal). The feature of multi-view data including multi-omics data is a data structure having signals from a plurality of different sources for one sample. For example, data of a set of a face photograph and a personal profile has two views of an “image” and “text”. In a case where the image and the text are merged and processed, a multi-view is obtained. The same applies to omics data, and it is assumed that data obtained by measuring gene expression data, methylation data, and protein expression data is present in each sample. In this case, each omics corresponds to a view. In a case where three omics are handled at the same time, the three omics are multi-omics.

### SUMMARY OF THE INVENTION

### [Tasks of Related Art]

In the technique described in “A robustness metric for biological data clustering algorithms”, Yuping Lu et al., [Searched on Sep. 9, 2022], Internet (https://pubmed.ncbi.nlm.nih.gov/31874625/), since the results of various settings are used for one algorithm, it is not possible to compare the setting values. For example, this technique is not used for determining an appropriate number of clusters (one of the setting values). In addition, in the technique disclosed in “A robustness metric for biological data clustering algorithms”, Yuping Lu et al., [Searched on Sep. 9, 2022], Internet (https://pubmed.ncbi.nlm.nih.gov/31874625/), it is not possible to perform evaluation related to the properties of the clusters. Since the evaluation is related to changes in a plurality of results, information related to the obtained clusters is not obtained. Further, biological information is not considered in the technique described in the “A robustness metric for biological data clustering algorithms”, Yuping Lu et al., [Searched on Sep. 9, 2022], Internet (https://pubmed.ncbi.nlm.nih.gov/31874625/). The results commonly obtained for various settings are not linked to biological robustness.

In addition, in the technique described in “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A:1023949509487), sub-sampling is repeated to output a plurality of cluster allocations. Therefore, there are samples that are not used for clustering. Further, in the technique described in “Consensus Clustering: A Resampling-Based Method for Class Discovery and Visualization of Gene Expression Microarray Data ”, Stefano Monti et al., [Searched on Sep. 9, 2022], Internet (https://link.springer.com/article/10.1023/A:1023949509487), execution is repeated on the essentially same sample population. Therefore, it is not possible to consider only one omics from which clusters are obtained. Therefore, in a case where there is a bias in a specific omics, clustering is affected by the bias. Further, in the technique described in “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A:1023949509487), biological information is not considered. Therefore, the results commonly acquired from a plurality of subsamples obtained from one omics are not linked to biological robustness.

In addition, in the technique described in “Multiplatform analysis of 12 cancer types reveals molecular classification within and across tissues of origin”, Katherine A Hoadley et al., [Searched on Sep. 9, 2022], Internet (https://pubmed.ncbi.nlm.nih.gov/25109877/), the result of executing clustering in each omics is stored in a binary vector and directly input to the “Consensus Cluster”. In this method, the internal criterion for the output cluster is not given. Therefore, a score for “stability” proposed in “Consensus Clustering” can be obtained, but this is a perspective on the non-determinism of the algorithm. There is no biological background.

[Tasks in Clustering of Omics Data]

Similarly to the general clustering analysis, the omics data clustering analysis is performed in the unsupervised setting, and there is a task for the evaluation based on the internal criterion or the like. In addition, since the omics data is obtained by measurement for a living organism, the omics data is characterized by having a property of including noise (temporal changes due to cellular heterogeneity and dynamics) derived from a biological reaction, in addition to noise related to the measurement. A biologically plausible (hereinafter, referred to as “biologically robust”) method that is less likely to be affected by the noise or the bias is a task in the clustering of omics data. However, the techniques in “A robustness metric for biological data clustering algorithms”, Yuping Lu et al., [Searched on Sep. 9, 2022], Internet (https://pubmed.ncbi.nlm.nih.gov/31874625/), “Consensus Clustering: A Resampling-Based Method for Class Discovery and Visualization of Gene Expression Microarray Data”, Stefano Monti et al., [Searched on Sep. 9, 2022], Internet (https://link.springer.com/article/10.1023/A:1023949509487), “Multiplatform analysis of 12 cancer types reveals molecular classification within and across tissues of origin”, Katherine A Hoadley et al., [Searched on Sep. 9, 2022], Internet (https://pubmed.ncbi.nlm.nih.gov/25109877/), and the like have not been able to sufficiently respond to the task.

The present invention has been made in view of the above circumstances, and an embodiment of the present invention provides an evaluation method and an evaluation device that can output an index indicating biological robustness of a cluster of multi-omics data. In addition, another embodiment of the present invention provides an evaluation method and an evaluation device that can output an index indicating biological robustness of a clustering result of multi-omics data. Further, still another embodiment of the present invention provides a visualization method and a visualization device that can visualize a result of evaluation by the evaluation method and the evaluation device.

According to a first aspect of the present invention, there is provided an evaluation method for a cluster executed by an evaluation device including a processor. The evaluation method comprises causing the processor to: acquire cluster allocation information obtained from clustering performed on multi-omics data consisting of two or more omics by any method independent for each omics; calculate a rate of match between cluster allocations of different omics for each cluster from the cluster allocation information; calculate a first quantitative index indicating biological robustness of the cluster for each cluster based on the rate of match; and output the calculated first quantitative index.

According to a second aspect, in the evaluation method according to the first aspect, the processor may be configured to: in the calculation of the rate of match, count the number of times each pair of samples belongs to the same cluster in the cluster allocation information of all of the clusters for all pairs of samples belonging to each cluster for each omics; multiply the number of times counted for each omics by a designated weight over all of the omics; calculate the rate of match for each cluster from a result of the weighting; and, in the calculation of the first quantitative index, calculate a first statistic for the rate of match for each cluster as the first quantitative index.

According to a third aspect, in the evaluation method according to the second aspect, the processor may be configured to calculate one or more of a sum, a mean, a median, a minimum value, a maximum value, and a mode of the number of times the weighting is performed as the first statistic.

According to a fourth aspect, in the evaluation method according to the second or third aspect, the processor may be configured to multiply the counted number of times by a designated constant to perform the weighting for each omics.

According to a fifth aspect, in the evaluation method according to the fourth aspect, the processor may be configured to perform the weighting while changing a constant by which some of the two or more omics are multiplied and a constant by which remaining omics are multiplied.

According to a sixth aspect, in the evaluation method according to any one of the second to fifth aspects, the processor may be configured to perform the weighting in a case where a result of the counting for any combination of omics is consistent with a designated condition and in a case where a result of the counting for any combination of samples is consistent with a designated condition.

According to a seventh aspect, in the evaluation method according to the first aspect, the processor may be configured to: in the calculation of the rate of match, calculate a similarity between each cluster of each omics and all of the clusters belonging to different omics; and calculate the first quantitative index, using a second statistic for the similarity as the rate of match.

According to an eighth aspect, in the evaluation method according to the seventh aspect, the processor may be configured to calculate one or more of a sum, a mean, a median, a minimum value, a maximum value, and a mode of the similarity as the second statistic.

According to a ninth aspect, in the evaluation method according to the seventh or eighth aspect, the processor may be configured to calculate any one of an inner product, a cosine similarity, a Jaccard coefficient, a Hamming distance, a Dice coefficient, or a correlation function between the clusters as the similarity.

According to a tenth aspect, in the evaluation method according to any one of the seventh to ninth aspects, the processor may be configured to weight each omics in a case where any combination of omics is consistent with a designated condition and any combination of samples is consistent with a designated condition in the calculation of the similarity.

According to an eleventh aspect, there is provided an evaluation method for a clustering result executed by an evaluation device including a processor. The evaluation method comprises causing the processor to: calculate the first quantitative index for each cluster using the evaluation method according to any one of the first to tenth aspects; calculate a third statistic of the first quantitative index; and output the third statistic as a second quantitative index indicating biological robustness of the clustering result.

According to a twelfth aspect, in the evaluation method according to the eleventh aspect, the processor may be configured to calculate any one of a sum, a mean, a median, a minimum value, a maximum value, or a mode of the first quantitative index as the third statistic.

A program that causes a computer to execute the evaluation method according to any one of the first to twelfth aspects and a non-transitory tangible recording medium in which a computer-readable code of the program is recorded can also be given as aspects of the present invention.

According to a thirteenth aspect, there is provided a visualization method for cluster evaluation executed by an evaluation device including a processor. The visualization method comprises causing the processor to: acquire cluster allocation information obtained from clustering performed on multi-omics data consisting of two or more omics by any method independent for each omics; represent each cluster extracted from each omics as a vector that has a sample belonging to each cluster as a component; compress the vector into three or less dimensions using any dimension compression method; plot each cluster at coordinates in the compressed dimensions; and output the first quantitative index calculated by the evaluation method according to any one of the first to twelfth aspects in association with each cluster.

According to a fourteenth aspect, there is provided a visualization method for cluster evaluation executed by an evaluation device including a processor. The visualization method comprises causing the processor to: construct a graph structure in which each cluster is a node and the similarity is an edge, based on the similarity calculated by the evaluation method according to the seventh aspect; and output the constructed graph structure and the first quantitative index calculated by the evaluation method according to any one of the first to tenth aspects in association with each cluster in the graph structure and the first quantitative index.

In addition, a program that causes a computer to execute the visualization method according to the thirteenth or fourteenth aspect and a non-transitory tangible recording medium in which a computer-readable code of the program is recorded can also be given as aspects of the present invention.

According to a fifteenth aspect, there is provided an evaluation device for a cluster. The evaluation device comprises a processor configured to: acquire cluster allocation information obtained from clustering performed on multi-omics data consisting of two or more omics by any method independent for each omics; calculate a rate of match between cluster allocations of different omics from the cluster allocation information; calculate a first quantitative index indicating biological robustness of the cluster for each cluster based on the rate of match; and output the calculated first quantitative index.

The evaluation device according to the fifteenth aspect may comprise a configuration that executes the same process as that in the second to tenth aspects.

According to a sixteenth aspect, in the evaluation device according to the fifteenth aspect, the processor may be configured to: in the calculation of the rate of match, count the number of times each pair of samples belongs to the same cluster in the cluster allocation information of all of the clusters for all pairs of samples belonging to each cluster for each omics; multiply the number of times counted for each omics by a designated weight over all of the omics; calculate the rate of match for each cluster from a result of the weighting; and in the calculation of the first quantitative index, calculate a first statistic for the rate of match for each cluster as the first quantitative index.

According to a seventeenth aspect, in the evaluation device according to the fifteenth aspect, the processor may be configured to: in the calculation of the rate of match, calculate a similarity between each cluster of each omics and all of clusters belonging to different omics; and calculate the first quantitative index, using a second statistic for the similarity as the rate of match.

According to an eighteenth aspect, there is provided an evaluation device for a clustering result. The evaluation device comprises a processor configured to: calculate the first quantitative index for each cluster using the evaluation device according to any one of the fifteenth to seventeenth aspects; calculate a third statistic of the first quantitative index; and output the third statistic as a second quantitative index indicating biological robustness of the clustering result.

The evaluation device according to the eighteenth aspect may comprise a configuration that executes the same process as that in the twelfth aspect.

According to a nineteenth aspect, there is provided a visualization device for cluster evaluation. The visualization device comprises a processor configured to: acquire cluster allocation information obtained from clustering performed on multi-omics data consisting of two or more omics by any method independent for each omics; represent each cluster extracted from each omics as a vector that has a sample belonging to each cluster as a component; compress the vector into three or less dimensions using any dimension compression method; plot each cluster at coordinates in the compressed dimensions; and output the first quantitative index calculated by the evaluation device according to any one of the fifteenth to seventeenth aspects in association with each cluster.

According to a twentieth aspect, there is provided a visualization device for cluster evaluation. The visualization device comprises a processor configured to: construct a graph structure in which each cluster is a node and the similarity is an edge, based on the similarity calculated by the evaluation device according to the seventeenth aspect; and output the constructed graph structure and the first quantitative index calculated by the evaluation device according to any one of the fifteenth to seventeenth aspects in association with each cluster in the graph structure and the first quantitative index.

### BRIEF DESCRIPTION OF THE DRAWINGS

FIGS. 1A and 1B are conceptual diagrams showing an aspect of clustering of multi-omics data.

FIG. 2 is a diagram showing a configuration of an evaluation device according to a first embodiment.

FIG. 3 is a diagram showing a configuration of a processing unit.

FIG. 4 is a flowchart showing a process of an evaluation method for a cluster.

FIG. 5 is a diagram showing an aspect in which a first quantitative index is calculated.

FIG. 6A to 6B2 are diagrams showing an aspect in which the counted number of times is weighted to calculate the first quantitative index.

FIG. 7 is a diagram showing an example of index calculation considering a similarity between clusters.

FIG. 8 is a flowchart showing a process of an evaluation method for a clustering result.

FIG. 9 is a diagram showing a functional configuration of a processing unit according to a second embodiment.

FIG. 10 is a flowchart showing a process of a visualization method for cluster evaluation.

FIG. 11 is a diagram showing an example of the visualization of the cluster evaluation.

FIG. 12 is another flowchart showing a process of the visualization method for cluster evaluation.

FIG. 13 is a diagram showing another example of the visualization of the cluster evaluation.

### DESCRIPTION OF THE PREFERRED EMBODIMENTS

Hereinafter, embodiments of an evaluation method, an evaluation device, a visualization method, and a visualization device according to the present invention will be described. In the description, the accompanying drawings will be referred to as necessary.

### First Embodiment

### [Concept of Clustering of Multi-Omics Data]

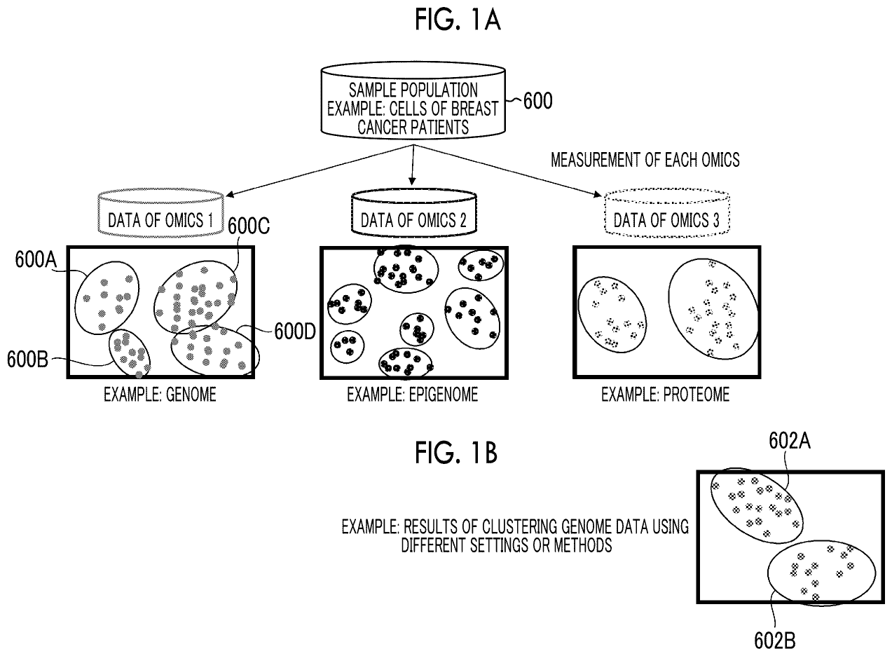

First, clustering of multi-omics data will be described with reference to a conceptual diagram of FIGS. 1A and 1B. In the example shown in FIGS. 1A and 1B, an aspect is shown in which a sample population is composed of, for example, data for cells of breast cancer patients and this data is measured for three omics (see FIG. 1A). Each omics data item is high-dimensional data (which has, for example, thousands to hundreds of thousands of feature amounts per sample).

In FIGS. 1A and 1B, a case where clustering is performed and then visualization using dimension compression is applied is assumed (which is the same as an example of visualization that will be described below). In this case, samples (for example, subtypes 600A to 600D) surrounded by circles in FIG. 1A indicate subtypes in breast cancer. Since the subtypes are used in the subsequent process, it is preferable to appropriately determine the subtypes.

However, for example, in a case where the subtypes are determined only on the basis of a genome (omics 1), there are four subtypes in the example shown in FIGS. 1A and 1B. However, in a case where clustering is performed by different settings or methods, there is a possibility that the number of subtypes will be, for example, two (subtypes 602A and 602B in FIG. 1B).

Here, attention is paid to other omics. Even though the omics are different, the omics are measurement results of the same sample population 600. Therefore, it is assumed that common properties are potentially present in the omics. Therefore, it is expected that a “biologically plausible subtype” can be defined by specifying a cluster commonly obtained in different omics (omics 2: epigenome, omics 3: proteome).

### [Configuration of Evaluation Device]

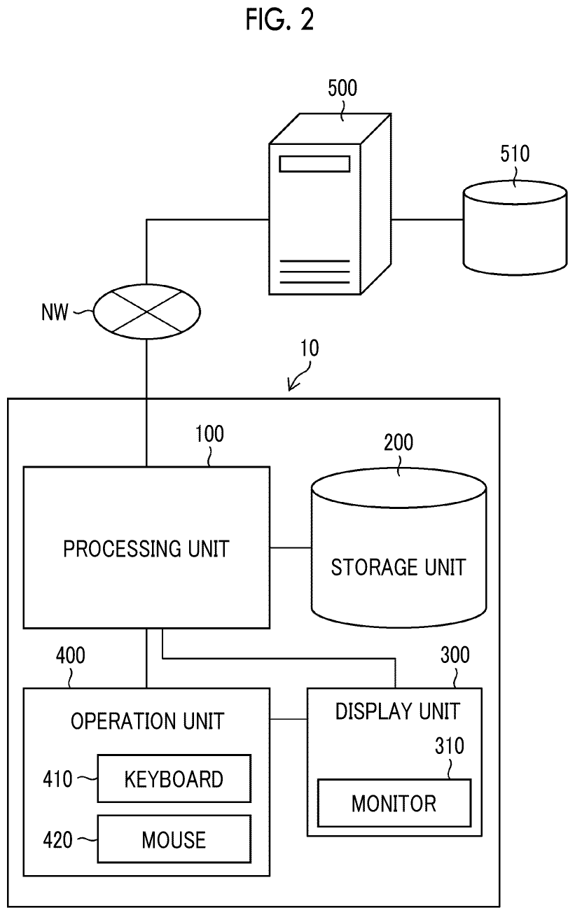

FIG. 2 is a diagram showing a schematic configuration of an evaluation device 10 (evaluation device) according to a first embodiment of the present invention. As shown in FIG. 2, the evaluation device 10 comprises a processing unit 100 (a processor or a computer), a storage unit 200, a display unit 300, and an operation unit 400, and these components are connected to each other to transmit and receive necessary information. These components can be installed in various forms. These components may be installed in one place (in one housing, one room, or the like), or these components may be installed in places separated from each other and may be connected via a network. In addition, the evaluation device 10 can be connected to an external server 500 and/or an external database 510 via a network NW, such as the Internet, and can perform the acquisition of multi-omics data, the reception of cluster allocation information, and the like as necessary. In addition, the evaluation device 10 can store processing results and the like in the external server 500 and/or the external database 510.

### [Configuration of Processing Unit]

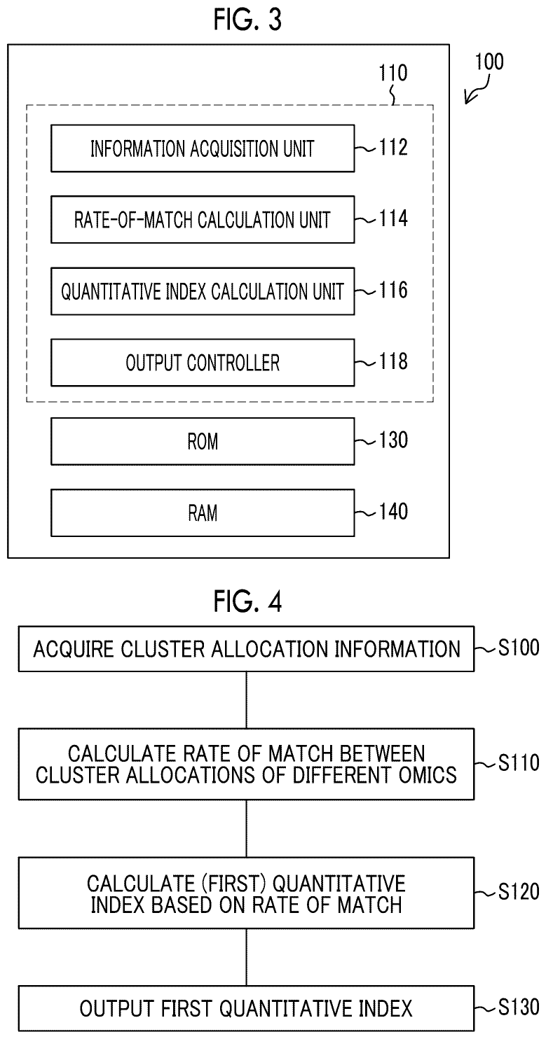

FIG. 3 is a diagram showing a configuration of the processing unit 100. As shown in FIG. 3, the processing unit 100 comprises a processor 110 (a processor or a computer), a read only memory (ROM) 130, and a random access memory (RAM) 140. The processor 110 performs the overall control of processes performed by each unit of the processing unit 100 and has functions of an information acquisition unit 112, a rate-of-match calculation unit 114, a quantitative index calculation unit 116, and an output controller 118. The functions are briefly described as follows. The information acquisition unit 112 (processor) acquires (receives or calculates) the cluster allocation information, the rate-of-match calculation unit 114 (processor) calculates a rate of match between cluster allocations of different omics from the cluster allocation information for each cluster, the quantitative index calculation unit 116 (processor) calculates a first quantitative index indicating biological robustness of the cluster for each cluster based on the rate of match, and the output controller 118 (processor) outputs the calculated first quantitative index and the like to the storage unit 200 and/or a monitor 310.

The information acquisition unit 112 can receive the multi-omics data or the cluster allocation information from the external server 500, the external database 510, or the like and/or from a recording medium, such as the storage unit 200, via the network NW. The output controller 118 can display the received or generated information, the calculation results (the first quantitative index, a second quantitative index, and the like), and the like on the monitor 310 and/or can store the information, the calculation results, and the like in the storage unit 200.

The functions of each unit of the processing unit 100 and the processor 110 can be implemented by various processors and the recording medium. The various processors include, for example, a central processing unit (CPU) which is a general-purpose processor that executes software (program) to implement various functions. In addition, the various processors also include a graphics processing unit (GPU), which is a processor specialized for image processing, and a programmable logic device (PLD) which is a processor whose circuit configuration can be changed after manufacturing, such as a field programmable gate array (FPGA). In a case where learning or recognition of the image is performed, a configuration using the GPU is effective. Further, the various processors also include a dedicated electric circuit which is a processor having a dedicated circuit configuration designed to execute a specific process, such as an application specific integrated circuit (ASIC).

The functions of each unit may be implemented by one processor or may be implemented by a plurality of processors of the same type or different types (for example, a plurality of FPGAs, a combination of the CPU and the FPGA, or a combination of the CPU and the GPU). In addition, a plurality of functions may be implemented by one processor. A first example of the configuration in which a plurality of functions are configured by one processor is an aspect in which one processor is configured by a combination of one or more CPUs and software and implements a plurality of functions. A representative example of this aspect is a computer. A second example of the configuration is an aspect in which a processor that implements the functions of the entire system using one integrated circuit (IC) chip is used. A representative example of this aspect is a system-on-chip (SoC). As described above, various functions are configured using one or more of the above-described various processors as a hardware structure. In addition, specifically, an electric circuit (circuitry) obtained by combining circuit elements, such as semiconductor elements, can be used as the hardware structure of the various processors. The electric circuit may be an electric circuit that implements the above-described functions using a logical sum, a logical product, a logical negation, an exclusive logical sum, and a logical operation of a combination thereof.

In a case where the processor or the electric circuit executes software (program), codes that can be read by a computer (for example, various processors or electric circuits constituting the processing unit 100 and/or a combination thereof) in the software to be executed are stored in a non-transitory tangible recording medium, such as the ROM 130, and the computer refers to the software. The software stored in the non-transitory tangible recording medium includes programs (an evaluation program and a visualization program) for executing the evaluation method and the visualization method according to the embodiment of the present invention and data (for example, multi-omics data, cluster allocation information, and setting values of hyperparameters and the like required for executing the programs) used in execution. The codes may be recorded in various recording media, such as magneto-optical recording devices and semiconductor memories, instead of the ROM 130. In a case of the processes using software, for example, the RAM 140 is used as a transitory storage area. In addition, data stored in a recording medium (not illustrated), such as an electronically erasable and programmable read only memory (EEPROM) or a flash memory, can also be referred to. The storage unit 200 may also be used as the “non-transitory tangible recording medium”.

Details of the processes using the processing unit 100 having the above-described configuration will be described below.

### [Configuration of Storage Unit]

The storage unit 200 is configured by various storage devices, such as a magneto-optical recording device, a hard disk, and a semiconductor memory, and a controller therefor and can store the multi-omics data, the cluster allocation information, the first quantitative index, the second quantitative index, the rate of match between the cluster allocations, the similarity between the clusters, and the like.

### [Configuration of Display Unit]

The display unit 300 comprises the monitor 310 (display device) that is configured by a display, such as a liquid crystal display, and can display input data, execution results of the evaluation method (evaluation program), the visualization method (visualization program), and the like. The monitor 310 may be configured by a touch panel display and may receive an instruction input by a user.

### [Configuration of Operation Unit]

The operation unit 400 comprises a keyboard 410 and a mouse 420, and the user can perform operations related to the execution of the evaluation method (evaluation program) and the visualization method (visualization program) according to the embodiment of the present invention, the display of the results, and the like through the operation unit 400. The operation unit 400 may comprise other operation devices.

### [Evaluation Method for Cluster]

An evaluation method for a cluster that is executed by the evaluation device 10 (processor) will be described in detail.

### [Acquisition of Cluster Allocation Information]

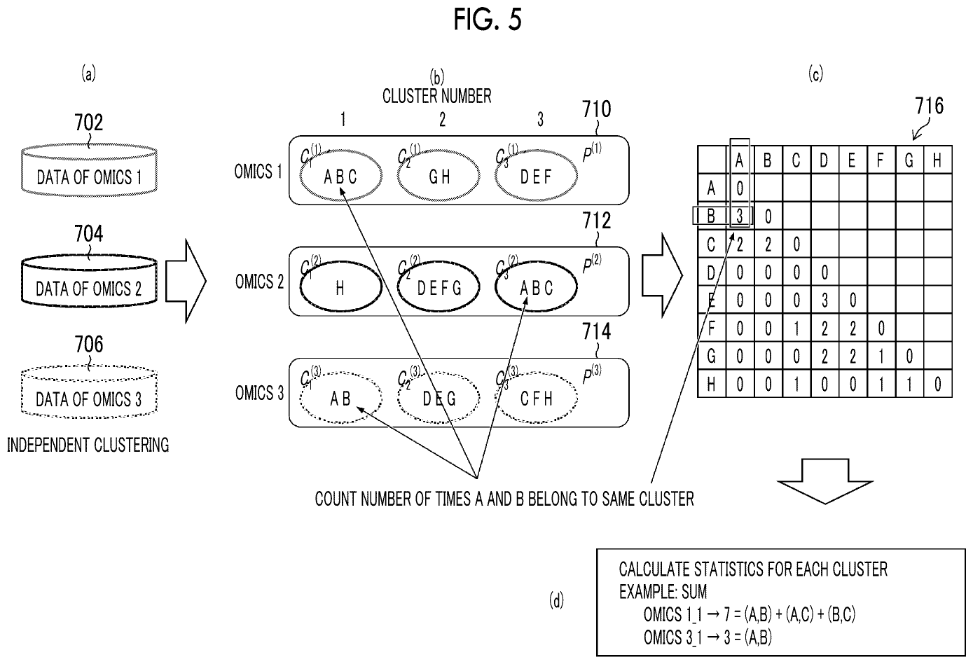

FIG. 4 is a flowchart showing a process of the evaluation method for a cluster. As shown in (a) of FIG. 5, the information acquisition unit 112 (processor) acquires the cluster allocation information obtained from the clustering executed on the multi-omics data consisting of two or more omics by any method independent for each omics (Step S100: an information acquisition step). In addition, in the embodiment of the present invention, the number of omics may be two or more, and there is no upper limit to the number of omics.

The “cluster allocation information” acquired in Step S100 is a result of the clustering executed on the multi-omics data by any method independent for each omics and holds information indicating “which cluster each sample is allocated to in each omics” as shown in (b) of FIG. 5. Further, the information acquisition unit 112 may receive the cluster allocation information calculated in advance from a recording device, such as the external server 500 or the external database 510. Alternatively, the information acquisition unit 112 may receive the multi-omics data from the recording device and perform cluster allocation to generate the cluster allocation information.

### [Calculation of Rate of Match]

The rate-of-match calculation unit 114 (processor) calculates the rate of match between the cluster allocations of different omics for each cluster from the acquired cluster allocation information (Step S110: a rate-of-match calculation step). The rate-of-match calculation unit 114 can count the number of times all pairs of samples belonging to each sample belong to the same cluster in the cluster allocation information of all of the clusters, multiplies the counted number of times by a designated weight, and calculate the rate of match for each cluster from the weighted results.

The number of samples is N, the number of omics is H, the number of clusters in omics h is K^{(h)} the number of samples belonging to an 1-th cluster Cₗ^{(h)} of omics h is Lₗ^{(h)}, and the cluster allocation for each omics h=1, 2, . . . , H is a set of clusters represented by the following Expression (4).

$${P}^{( h )} = \{ {C}_{1}^{( h )} , {C}_{2}^{( h )} , … , {C}_{{K}^{( h )}}^{( h )} \} \qquad ( 4 )$$

Here, the 1-th cluster is a set of samples represented by the following Expression (5).

$${C}_{l}^{( h )} = \{ {s}_{l , 1}^{( h )} , {s}_{l , 2}^{( h )} , … , {s}_{l , {L}_{l}^{( h )}}^{( h )} \} \qquad ( 5 )$$

where S_{l,1}^{(h)} is a sample number.

In this case, the matrix M^{(h)} indicating whether or not each sample belongs to the same cluster is defined by the following Expression (6).

$${M}^{( h )} ( i , j ) = \{ \begin{matrix}1 if i , j ∈ D , where D ∈ {P}^{( h )} \\ 0 otherwise\end{matrix} \qquad ( 6 )$$

In a case where (i, j) is a set of the i-th sample and the j-th sample, the matrix M^{(h)} is an N×N dimensional matrix.

Therefore, the rate-of-match calculation unit 114 can calculate the rate of match between the cluster allocations in all of the omics using the following Expression (7) (Step S110).

$$M ( i , j ) = {\sum }_{h = 1}^{H} {M}^{( h )} \qquad ( 7 )$$

The matrix M^{(h)} indicates the count of the number of times each sample pair belongs to the same cluster for each omics, and the sum (Σ) of the matrix M^{(h)} for h (=1 to H) means that the sum over all of omics is calculated. In addition, in Expression (7), weights for each omics are equal, that is, are set to 1. However, M^{(h)} may be multiplied by a designated weight, which will be described below.

In (c) of FIG. 5, the table 716 is an example of the representation of the rate of match (matrix M^{(h)}). Further, in the table 716, an upper triangle is symmetrical to a lower triangle.

Furthermore, the quantitative index calculation unit 116 (processor) calculates biological robustness Rₗ^{(h)} of each cluster as the first quantitative index (an index indicating the biological robustness of the cluster) using the following Expression (8) (Step S120: a first quantitative index calculation step). The biological robustness Rₗ^{(h)} is a statistic of the rate of match for each cluster.

$${R}_{l}^{( h )} = F ( \{ M ( i , j ) \vert ( i , j ) ∈ {C}_{l}^{( h )} \} ) \qquad ( 8 )$$

Here, F(·) is a function that takes a set as an argument and returns a scalar. For the weighted count M(i, j), one or more any statistics (first statistics), such as a sum, a mean, a median, a minimum value, a maximum value, and a mode, can be used. In addition, (d) of FIG. 5 and Expression (8) show an example of calculation in which a summatory function is used for F. However, the quantitative index calculation unit 116 may calculate the first quantitative index using another function (another statistic) such as the mean of the rate of match.

The quantitative index calculation unit 116 can determine which statistic is used to calculate the first quantitative index in response to the operation of the user through the keyboard 410 and/or the mouse 420 (operation unit 400). The quantitative index calculation unit 116 may determine the statistic to be used, without depending on the operation of the user.

The output controller 118 (processor) outputs the calculated first quantitative index (Step S130: an index output step). The first quantitative index is an index indicating the biological robustness of the cluster. As the value of the index is larger, the biological robustness of the cluster is higher. The output controller 118 may store the first quantitative index in the storage unit 200 or may display the first quantitative index on the monitor 310 (display device). The display on the monitor 310 can be performed, for example, by characters, numbers, figures, symbols, or combinations thereof indicating the index, and the colors of the characters, the numbers, the figures, the symbols, and the like may be changed depending to the value of the index. In addition, it is preferable that the output controller 118 outputs the cluster and the first quantitative index for the cluster in association with each other.

In the first embodiment, the biological robustness of the cluster of the multi-omics data can be evaluated by the first quantitative index. The same applies to each of the following aspects.

### [Weight of Count]

In the above-described aspect, weights for each omics are equal, that is, are set to 1 (in Expression (7), all of the weights for the matrix M^{(h)} are 1). However, in the embodiment of the present invention, the weight for the counted number of times may be changed depending on the omics or a combination thereof. This aspect will be described below.

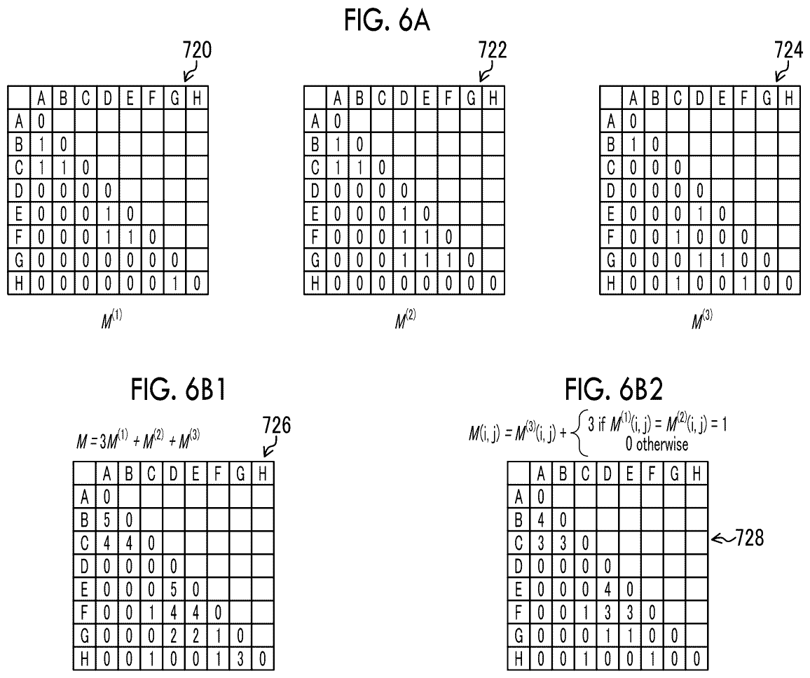

FIGS. 6A to 6B2 are diagrams showing an aspect in which the counted number of times is weighted to calculate the first quantitative index. The information acquisition unit 112 (processor) acquires the cluster allocation information of each omics (Step S100: an information acquisition step). FIG. 6A shows an example of the cluster allocation information of omics 1 to 3 (tables 720 to 724). In addition, in the tables 720 to 724 and tables 726 and 728, an upper triangle is symmetrical to a lower triangle (which is the same as that in the table 716 shown in FIG. 5).

### [Aspect 1 of Weighting]

In the above-described Expression (7), the weights for each omics are equal, that is, are set to 1. However, in aspect 1, the weights for each omics are changed. Specifically, the rate-of-match calculation unit 114 can perform weighting, for example, using the following Expression (9).

$$M ( i , j ) = 3 {M}^{( 1 )} + {M}^{( 2 )} + {M}^{( 3 )} \qquad ( 9 )$$

In Expression (9), the rate-of-match calculation unit 114 multiplies the counted number of times (M^{(h)}) by a constant designated for each omics (3 for omics 1, and 1 for omics 2 and 3) to perform weighting. As described above, the rate-of-match calculation unit 114 can perform weighting while changing a constant by which some of two or more omics are multiplied and a constant by which the remaining omics are multiplied.

The results of the weighting by Expression (9) are as shown in the table 726 in (b) of FIG. 6. Then, the weighting by Expression (9) can be generalized as represented by the following Expression (10). Here, w is an H-dimensional real vector that is given to each omics (w₁, w₂, and w₃ are coefficients designated for each omics).

$$M ( i , j ) = w [ \begin{matrix}{M}^{( 1 )} \\ {M}^{( 2 )} \\ {M}^{( 3 )}\end{matrix} ] = [ \begin{matrix}{w}_{1} & {w}_{2} & {w}_{3}\end{matrix} ] [ \begin{matrix}{M}^{( 1 )} \\ {M}^{( 2 )} \\ {M}^{( 3 )}\end{matrix} ] \qquad ( 10 )$$

The quantitative index calculation unit 116 can calculate the first quantitative index from the weighted results using the above-described Expression (8) (Step S120: a first quantitative index calculation step), and the output controller 118 can output the calculated first quantitative index (Step S130: an index output step). As in the description of Expression (8), even for the statistic (first statistic) for the result of the weighting, one or more of any statistics, such as a sum, a mean, a median, a minimum value, a maximum value, and a mode, can be used. The type of statistic to be used corresponds to the type of function F to be used in Expression (8) (the same applies to the following aspects).

### [Aspect 2 of Weighting]

In aspect 2, the rate-of-match calculation unit 114 multiplies a weight of 3 only in a case where the results of the omics 1 and the omics 2 are consistent with each other, as shown in Expression (11).

$$M ( i , j ) = {M}^{( 3 )} ( i , j ) + \{ \begin{matrix}3 if {M}^{( 1 )} ( i , j ) = {M}^{( 2 )} ( i , j ) = 1 \} \\ 0 otherwise\end{matrix} \qquad ( 11 )$$

The condition “in a case where the results of the omics 1 and the omics 2 are consistent with each other” and the numerical value “three times” are examples of the weighting, and other conditions and numerical values may be used. The results of the weighting by Expression (11) are as shown in the table 728 in FIG. 6B2. Further, in aspect 2, the calculation and output of the first quantitative index can be performed by Expression (8) in the same manner as in aspect 1.

### [Other Aspects of Weighting]

The above-described aspects 1 and 2 are examples of the weighting, and the rate-of-match calculation unit 114 may perform weighting, using “the condition is that the results in three or more omics are consistent with each other” (an aspect in which “weighting is performed in a case where a counting result for any combination of omics is consistent with a designated condition”) or “weighting is performed only in a case where results for a specific sample pair are consistent in a specific omics” (an aspect in which “weighting is performed in a case where a counting result for any combination of samples is consistent with a designated condition”), in addition to the above. Further, a weighting method may be “addition of the weight” instead of the multiplication of the weight. The quotient or difference can be taken by setting a weight that is less than 1 or less than 0.

### [Aspect in Which Statistic of Similarity Between Clusters is Calculated as Index]

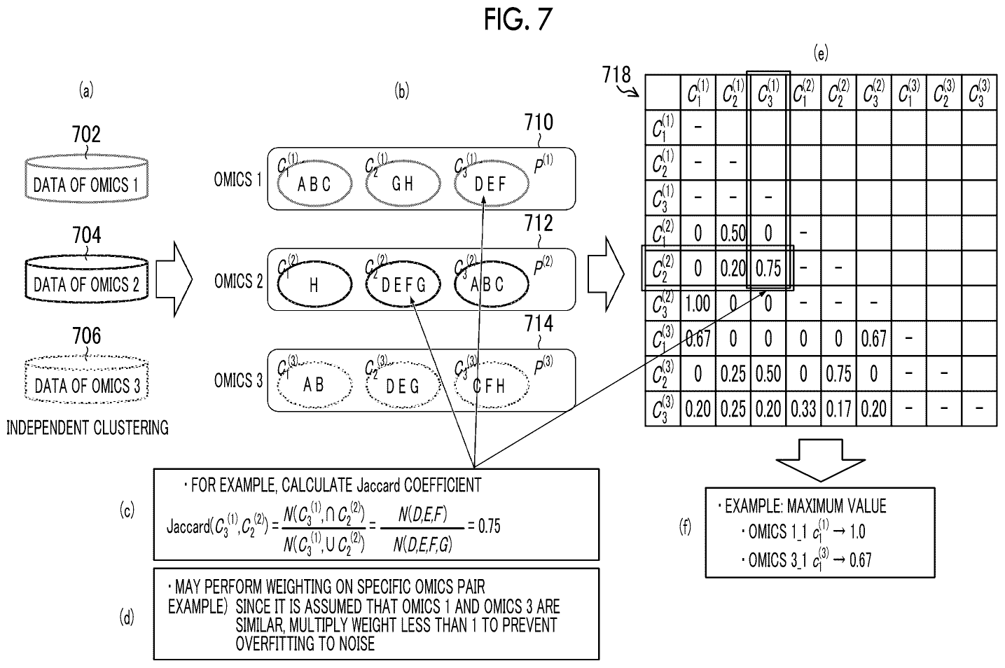

In the embodiment of the present invention, in the calculation of the rate of match, the rate-of-match calculation unit 114 (processor) can calculate the similarity between each cluster of each omics and all of the clusters belonging to different omics, and the quantitative index calculation unit 116 (processor) can calculate the first quantitative index, using a second statistic for the similarity as the rate of match. FIG. 7 is a diagram showing an example of index calculation considering the similarity between the clusters.

The information acquisition unit 112 can acquire the cluster allocation information as in Step S100 shown in FIG. 4 ((a) and (b) of FIG. 7), and the rate-of-match calculation unit 114 calculates the similarity between each cluster of each omics and all of the clusters belonging to different omics. The rate-of-match calculation unit 114 can calculate, for example, any of an inner product, a cosine similarity, a Jaccard coefficient, a Hamming distance, a Dice coefficient, and a correlation function between the clusters as the similarity. However, the similarity is not limited to these examples.

(c) of FIG. 7 shows a result of calculating a Jaccard coefficient (an example of the similarity) for C₃⁽¹⁾ (cluster 3 of omics 1) and C₂⁽²⁾ (cluster 2 of omics 2). In this case, the similarity is 0.75. In addition, (e) of FIG. 7 is a table showing the results of calculating the Jaccard coefficients for omics 1 to 3 and clusters 1 to 3.

The quantitative index calculation unit 116 can calculate the first quantitative index, using, as the rate of match, the second statistic for the similarity calculated in this way. In the example shown in (f) of FIG. 7, the maximum value (an example of the second statistic) of the similarity is used as the rate of match. In this example, for C₁⁽¹⁾ (cluster 1 of omics 1), 1.0 which is the maximum value (an example of the second statistic) of {0.0, 0.0, 1.0, 0.67, 0.0, 0.2} is the rate of match. Similarly, for C₁⁽³⁾ (cluster 1 of omics 3), 0.67 which is the maximum value (an example of the second statistic) of {0.67, 0.0, 0.0, 0.0, 0.0, 0.67} is the rate of match. The quantitative index calculation unit 116 calculates the rate of match as the first quantitative index indicating the biological robustness of the cluster.

A large value of the first quantitative index means that the biological robustness of the cluster is high. In the above-described example, the robustness (1.0) of cluster 1 of omics 1 is higher than the robustness (0.67) of cluster 1 of omics 3.

### [Weighting of Each Omics in Calculation of Similarity]

The rate-of-match calculation unit 114 may weight each omics in the above-described calculation of the similarity. For example, as described in (d) of FIG. 7, in a case where common noise or the like is known in a specific omics pair (in the example shown in FIG. 7, a pair of omics 1 and omics 3), a weight that is less than 1 can be multiplied to prevent overfitting to noise. Conversely, in a case where it is determined that the reliability of the rate of match in a specific omics pair (in the example shown in FIG. 6B2, a pair of omics 1 and omics 2) is increased as in the example shown in FIG. 6B2, a weight that is greater than 1 may be multiplied or added.

### [Evaluation Method for Clustering Result]

In the above-described evaluation method for a cluster, one index (first quantitative index) is given for each cluster. In the embodiment of the present invention, one or more statistics can be calculated from the first quantitative index for each cluster to calculate a quantitative index for the biological robustness of the clustering result, which will be described below.

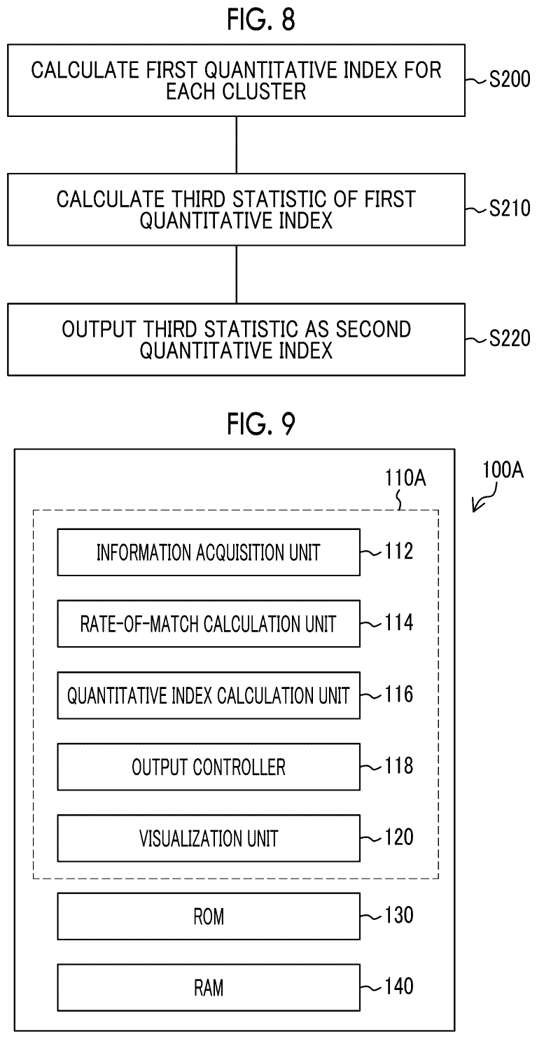

FIG. 8 is a flowchart showing a process in the evaluation method for a clustering result. Each unit of the processing unit 100 (processor) calculates the first quantitative index for each cluster in the same manner as described in the evaluation method for a cluster (Step S200: an information acquisition step, a rate-of-match calculation step, an index calculation step, and an index output step). The process in Step S200 is the same as the process in Steps S100 to S120 shown in FIG. 4, and a detailed description thereof will be omitted. Further, in the process in Step S200, any of the variations described in Steps S100 to S120 can be used.

The quantitative index calculation unit 116 (processor) calculates any one of the sum, mean, median, minimum value, maximum value, or mode of the first quantitative index as a third statistic (Step S210: a statistic calculation step). The quantitative index calculation unit 116 can determine the statistic to be calculated, in response to the operation of the user through the operation unit 400, or can automatically perform the determination without depending on the operation of the user. The output controller 118 (processor) outputs the third statistic as the second quantitative index indicating the biological robustness of the clustering result (that is, the clustering method or a clustering setting value set) (Step S220: an index output step).

### Second Embodiment

### [Visualization of Cluster Evaluation (Aspect 1)]

In the embodiment of the present invention, the cluster evaluation (the evaluation of the biological robustness of the cluster or the evaluation of the cluster allocation) can be visualized using the index described in the first embodiment. FIG. 9 is a diagram showing a configuration of a processing unit 100A (processor) of an evaluation device 10 (evaluation device) according to a second embodiment. The processing unit 100A differs from the processing unit 100 (see FIG. 3) according to the first embodiment in that it further comprises a visualization unit 120 (processor). Other configurations of the evaluation device 10 according to the second embodiment are the same as those of the evaluation device 10 according to the first embodiment.

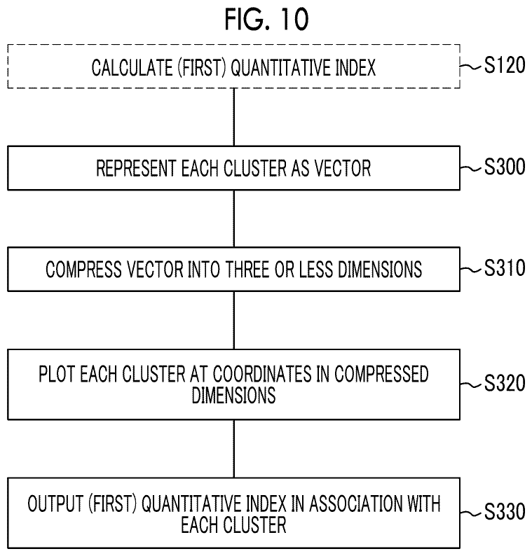

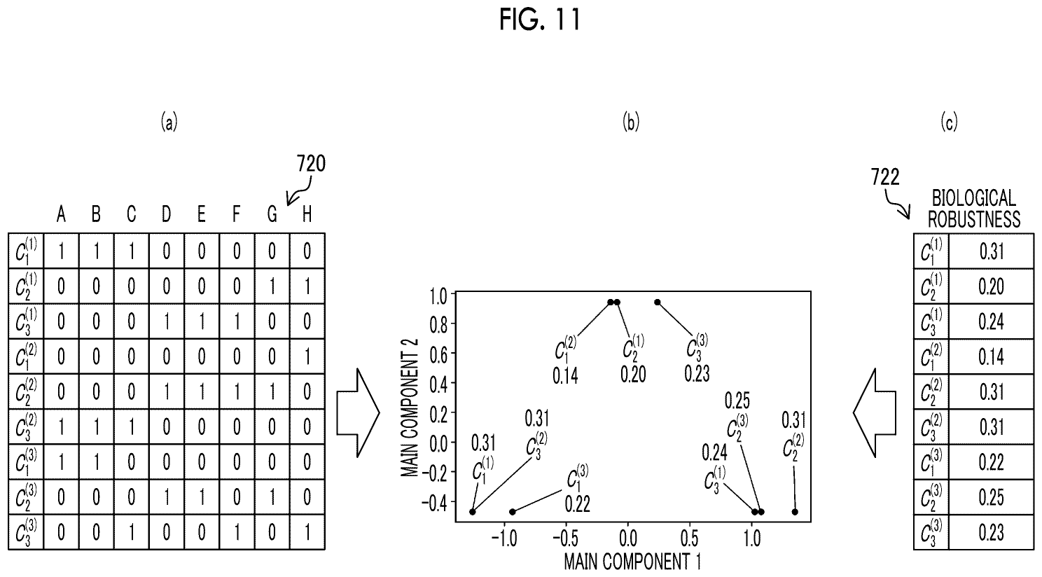

FIG. 10 is a flowchart showing a process of a visualization method for cluster evaluation (aspect 1), and FIG. 11 is a diagram showing an aspect of the visualization of the cluster evaluation. In the flowchart shown in FIG. 10, the calculation of the first quantitative index is the same as that in the first embodiment (see Steps S100 to S120 shown in FIG. 4), and the information acquisition unit 112 acquires the cluster allocation information.

In the visualization of the cluster evaluation, the visualization unit 120 (processor) represents each cluster extracted from each omics as a vector that has a sample belonging to each cluster as a component (Step S300: a visualization step). For example, as shown in (a) (table 720) of FIG. 11, C₁⁽¹⁾ (cluster 1 of omics 1)=(1, 1, 1, 0, 0, 0, 0, 0) indicates that samples A, B, and C are included in C₁⁽¹⁾. In addition, in a case where this sequence is regarded as a vector, the rate-of-match calculation unit 114 (processor) can calculate the similarity between two clusters using the distance (the cosine distance, the Hamming distance or the like) of the vector.

The visualization unit 120 compresses the obtained vector into three or less dimensions using any dimension compression method (Step S310: a dimension compression step) and plots each cluster at coordinates in the compressed dimensions (Step S320: a plotting step). The output controller 118 further outputs the first quantitative index calculated by the same method as in the first embodiment in association with each plotted cluster (Step S330: an output step).

(b) of FIG. 11 is a diagram showing a state in which the above-described vectors are compressed into two dimensions by principal component analysis. As can be seen from (b) of FIG. 11, each point indicates the cluster, and similar clusters are plotted at close coordinates. The principal component analysis is a method that compresses multidimensional data (here, the vector) into a low dimension while preserving information as much as possible. Further, examples of the dimension compression method include a uniform manifold approximation and projection (UMAP) method and a t-distributed stochastic neighbor embedding (t-SNE) method in addition to the principal component analysis. It is preferable that the visualization unit 120 selects a dimension compression method suitable for the properties of the data. In addition, the visualization unit 120 may select the dimension compression method in response to the operation of the user through the operation unit 400.

(c) of FIG. 11 is a table (table 722) showing the first quantitative index for each cluster of each omics. In (b) of in FIG. 11, the first quantitative index included in this table is displayed in the vicinity of the plot of each cluster (an example of association output). The output controller 118 may display the plotting results on the monitor 310 (display device) or may store the plotting results in the storage unit 200. A diagram of the plotting results may be output by a printer (not shown). This visualization enables the user to easily ascertain the biological robustness of the cluster.

### [Visualization of Cluster Evaluation (Aspect 2)]

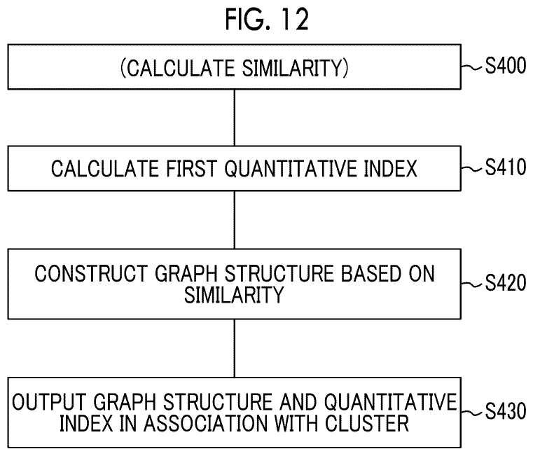

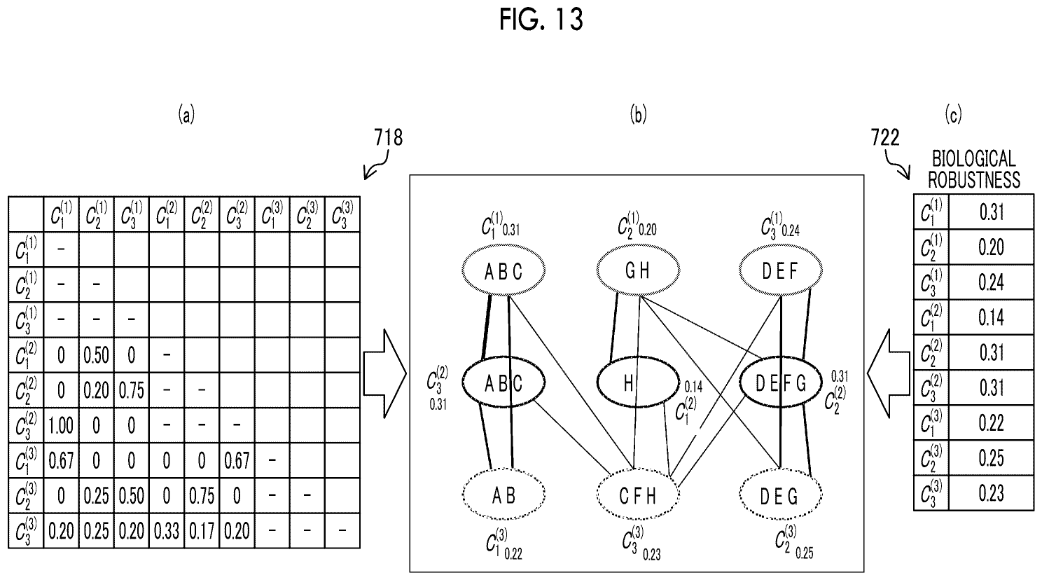

FIG. 12 is a flowchart showing a process of a visualization method for cluster evaluation (aspect 2), and FIG. 13 is a diagram showing an aspect of the visualization of the cluster evaluation. In the flowchart shown in FIG. 12, each unit of the processing unit 100 (processor) can calculate the first quantitative index as in the first embodiment (see Steps S100 to S120 shown in FIG. 4; Step S410). In addition, each unit of the processing unit 100 (processor) can calculate the similarity (Step S400) in the same manner as described above with reference to FIG. 7.

The visualization unit 120 constructs a graph structure, in which each cluster is a node and the similarity is an edge, based on the similarity calculated in Step S400 (Step S420: a graph structure construction step), and the output controller 118 outputs the constructed graph structure and the first quantitative index in association with each cluster in the graph structure and the first quantitative index (Step S430: an output step).

FIG. 13 is a diagram showing an aspect of the visualization according to aspect 2. (a) of FIG. 13 is a table (a table 718; which is the same as that shown in (e) of FIG. 7) showing the similarity, and (b) of FIG. 13 is a diagram showing the graph structure. In FIG. 13, the first quantitative index (which is the same as that shown in (b) of FIG. 11) shown in (c) of FIG. 13 is output in the vicinity of each cluster. In addition, in the diagram ((b) of FIG. 13) of the graph structure, the thickness of the edge indicates the magnitude of the similarity. The output controller 118 may perform an emphasis process, such as a process of changing the size of a symbol (an ellipse in the diagram) indicating the cluster according to the size of the first quantitative index or a process of changing the line type or color of the symbol, or may perform an emphasis process, such as a process of changing the line type or color of the edge according to the magnitude of the similarity, in the diagram of the graph structure. In addition, the output controller 118 may display the diagram of the constructed graph structure on the monitor 310 (display device) or may store the diagram of the constructed graph structure in the storage unit 200. The diagram may be output by the printer (not shown). This visualization also enables the user to easily ascertain the biological robustness of the cluster.

The embodiments of the present invention have been described above. However, the present invention is not limited to the above-described aspects and can be modified in various ways without departing from the gist of the present invention.

### Explanation of References

10: evaluation device
100: processing unit
100A: processing unit
110: processor
112: information acquisition unit
114: rate-of-match calculation unit
116: quantitative index calculation unit
118: output controller
120: visualization unit
130: ROM
140: RAM
200: storage unit
300: display unit
310: monitor
400: operation unit
410: keyboard
420: mouse
500: external server
510: external database
600: sample population
600A: subtype
600B: subtype
600C: subtype
600D: subtype
602A: subtype
602B: subtype
NW: network
S100 to S220: each step of evaluation method
S300 to S330: each step of visualization method
S400 to S430: each step of visualization method

What is claimed is:

1. An evaluation method for a cluster executed by an evaluation device including a processor, the evaluation method comprising:
causing the processor to:
acquire cluster allocation information obtained from clustering performed on multi-omics data consisting of two or more omics by any method independent for each omics;
calculate a rate of match between cluster allocations of different omics for each cluster from the cluster allocation information;
calculate a first quantitative index indicating biological robustness of the cluster for each cluster based on the rate of match; and
output the calculated first quantitative index.

2. The evaluation method according to claim 1, wherein the processor is configured to:
in the calculation of the rate of match,
count the number of times each pair of samples belongs to the same cluster in the cluster allocation information of all of the clusters for all pairs of samples belonging to each cluster for each omics;
multiply the number of times counted for each omics by a designated weight over all of the omics;
calculate the rate of match for each cluster from a result of the weighting; and
in the calculation of the first quantitative index,
calculate a first statistic for the rate of match for each cluster as the first quantitative index.

3. The evaluation method according to claim 2, wherein the processor is configured to:
calculate one or more of a sum, a mean, a median, a minimum value, a maximum value, and a mode of the number of times the weighting is performed as the first statistic.

4. The evaluation method according to claim 2, wherein the processor is configured to:
multiply the counted number of times by a designated constant to perform the weighting for each omics.

5. The evaluation method according to claim 4, wherein the processor is configured to:
perform the weighting while changing a constant by which some of the two or more omics are multiplied and a constant by which remaining omics are multiplied.

6. The evaluation method according to claim 2, wherein the processor is configured to:
perform the weighting in a case where a result of the counting for any combination of omics is consistent with a designated condition and in a case where a result of the counting for any combination of samples is consistent with a designated condition.

7. The evaluation method according to claim 1, wherein the processor is configured to:
in the calculation of the rate of match, calculate a similarity between each cluster of each omics and all of the clusters belonging to different omics; and
calculate the first quantitative index, using a second statistic for the similarity as the rate of match.

8. The evaluation method according to claim 7, wherein the processor is configured to:
calculate one or more of a sum, a mean, a median, a minimum value, a maximum value, and a mode of the similarity as the second statistic.

9. The evaluation method according to claim 7, wherein the processor is configured to:
calculate any one of an inner product, a cosine similarity, a Jaccard coefficient, a Hamming distance, a Dice coefficient, or a correlation function between the clusters as the similarity.

10. The evaluation method according to claim 7, wherein the processor is configured to:
weight each omics in a case where any combination of omics is consistent with a designated condition and any combination of samples is consistent with a designated condition in the calculation of the similarity.

11. An evaluation method for a clustering result executed by an evaluation device including a processor, the evaluation method comprising:
causing the processor to:
calculate the first quantitative index for each cluster using the evaluation method according to claim 1;
calculate a third statistic of the first quantitative index; and
output the third statistic as a second quantitative index indicating biological robustness of the clustering result.

12. The evaluation method according to claim 11, wherein the processor is configured to:
calculate any one of a sum, a mean, a median, a minimum value, a maximum value, or a mode of the first quantitative index as the third statistic.

13. A visualization method for cluster evaluation executed by an evaluation device including a processor, the visualization method comprising:
causing the processor to:
acquire cluster allocation information obtained from clustering performed on multi-omics data consisting of two or more omics by any method independent for each omics;
represent each cluster extracted from each omics as a vector that has a sample belonging to each cluster as a component;
compress the vector into three or less dimensions using any dimension compression method;
plot each cluster at coordinates in the compressed dimensions; and
output the first quantitative index calculated by the evaluation method according to claim 1 in association with each cluster.

14. A visualization method for cluster evaluation executed by an evaluation device including a processor, the visualization method comprising:
causing the processor to:
construct a graph structure in which each cluster is a node and the similarity is an edge, based on the similarity calculated by the evaluation method according to claim 7;
acquire cluster allocation information obtained from clustering performed on multi-omics data consisting of two or more omics by any method independent for each omics;
calculate a rate of match between cluster allocations of different omics for each cluster from the cluster allocation information;
calculate a first quantitative index indicating biological robustness of the cluster for each cluster based on the rate of match; and
output the calculated first quantitative index and the constructed graph structure, in association with each cluster in the graph structure and the first quantitative index.

15. An evaluation device for a cluster, the evaluation device comprising:
a processor configured to:
acquire cluster allocation information obtained from clustering performed on multi-omics data consisting of two or more omics by any method independent for each omics;
calculate a rate of match between cluster allocations of different omics from the cluster allocation information;
calculate a first quantitative index indicating biological robustness of the cluster for each cluster based on the rate of match; and
output the calculated first quantitative index.

16. The evaluation device according to claim 15, wherein the processor is configured to:
in the calculation of the rate of match, count the number of times each pair of samples belongs to the same cluster in the cluster allocation information of all of the clusters for all pairs of samples belonging to each cluster for each omics;
multiply the number of times counted for each omics by a designated weight over all of the omics;
calculate the rate of match for each cluster from a result of the weighting; and
in the calculation of the first quantitative index, calculate a first statistic for the rate of match for each cluster as the first quantitative index.

17. The evaluation device according to claim 15, wherein the processor is configured to:
in the calculation of the rate of match, calculate a similarity between each cluster of each omics and all of clusters belonging to different omics; and
calculate the first quantitative index, using a second statistic for the similarity as the rate of match.

18. An evaluation device for a clustering result, the evaluation device comprising:
a processor configured to:
calculate the first quantitative index for each cluster using the evaluation device according to claim 15;
calculate a third statistic of the first quantitative index; and
output the third statistic as a second quantitative index indicating biological robustness of the clustering result.

19. A visualization device for cluster evaluation, the visualization device comprising:
a processor configured to:
acquire cluster allocation information obtained from clustering performed on multi-omics data consisting of two or more omics by any method independent for each omics;
represent each cluster extracted from each omics as a vector that has a sample belonging to each cluster as a component;
compress the vector into three or less dimensions using any dimension compression method;
plot each cluster at coordinates in the compressed dimensions; and
output the first quantitative index calculated by the evaluation device according to claim 15 in association with each cluster.

20. A visualization device for cluster evaluation, the visualization device comprising:
a processor configured to:
construct a graph structure in which each cluster is a node and the similarity is an edge, based on the similarity calculated by the evaluation device according to claim 17;
acquire cluster allocation information obtained from clustering performed on multi-omics data consisting of two or more omics by any method independent for each omics;
calculate a rate of match between cluster allocations of different omics from the cluster allocation information;
calculate a first quantitative index indicating biological robustness of the cluster for each cluster based on the rate of match; and
output the calculated first quantitative index and the constructed graph structure, in association with each cluster in the graph structure and the first quantitative index.
